(19) United States
(12) Patent Application Publication
(10) Pub. No.: US 2014/0217623 A1
(43) Pub. Date: Aug. 7, 2014

(54) SCENT DELIVERY METHOD AND APPARATUS USING AN EXISTING AIR MOVING DEVICE

(71) Applicant: Helen of Troy Limited, St. Michael (BB)

(72) Inventor: John Franks, Hopkinton, MA (US)

(73) Assignee: Helen of Troy Limited, St. Michael (BB)

(21) Appl. No.: 14/249,933

(22) Filed: Apr. 10, 2014

Related U.S. Application Data
(62) Division of application No. 13/344,048, filed on Jan. 5, 2012.
(60) Provisional application No. 61/430,080, filed on Jan. 5, 2011.

(51) Int. Cl. A61L 9/12

(52) U.S. Cl. CPC A61L 9/122
USPC 261/31; 29/428

(57) ABSTRACT

A scent-releasing mechanism that is usable with an existing air moving device. The scent-releasing mechanism includes a chamber configured to enclose a scent source such as a scent cartridge, scent pad or the like, an air inlet, and an air outlet to release scent into a stream of air moved by the air moving device, so that the scent is dispersed throughout the room. At least one of the air inlet or air outlet is an adjustable port. The adjustable port may be adjusted to control the amount of scent released through the air outlet.

### CROSS-REFERENCE TO RELATED APPLICATIONS

This application claims the benefit of U.S. Provisional Application No. 61/430,080, filed Jan. 5, 2011, the contents of which are incorporated herein by reference.

### FIELD OF THE INVENTION

The present invention generally relates to a scent-releasing mechanism and related method that is usable with an air moving device.

BACKGROUND OF INVENTION

The ability to easily and efficiently control indoor environments is highly desirable. For this reason, a number of devices have been developed to control the temperature, humidity, odor and air quality of enclosed environments such as the rooms of a house. These mechanisms may rely upon diffusion, convection, or an assisting device such as a fan to move air in order to better control the indoor environment in a room. For instance, during the winter months, a lack of humidity in the air can be a source of significant discomfort to people. Thus, humidifiers are a typical device used to increase or control the humidity in the room.

Furthermore, during these same months, many people develop colds and may have sinus and chest congestion. One method for helping with the symptoms associated with congestion and colds is by dispersing medicinal vapors in the air, such as menthol or the like. The medicinal vapors help reduce cold symptoms as well as sinus and chest congestion. Medicinal vapors can be released and dispersed by scent generators, similar to air fresheners. Scents may also be released and dispersed for non-medicinal purposes, such as for providing a pleasing scent or masking an unpleasant odor. It is desirable that the scent should be released only when needed, in order to avoid waste of the scent.

Users may already have a humidifier, or other air moving device, to control the room environment when they become aware of their need to release and disperse scents into the air. Although a separate device may be used to disperse the scent, having another device—in addition to the device(s) to control the room environment—may cause problems. For instance, the number of outlets in the room may be limited. Further, the number of devices needed to control all of the desired environmental characteristics in a room can result in an unsightly mess and crowding of the room.

Thus, there is a need for a device that can combine or retrofit a scent-releasing mechanism with an existing air moving device, which is simpler and lower cost than existing designs.

### SUMMARY OF THE INVENTION

Embodiments of the present invention provide a scent-releasing mechanism for an air moving device, and specifically for an existing air-moving device. The scent-releasing mechanism includes a chamber configured to enclose a scent source such as a scent cartridge, scent pad or the like, an air inlet, and an air outlet to release scent into a stream of air moved by the air moving device, so that the scent is dispersed throughout the room. At least one of the air inlet or air outlet is an adjustable port. The adjustable port may be adjusted to control the amount of scent released through the air outlet.

In a specific embodiment of the invention a scent-releasing appliance includes a housing, an air-moving device disposed within the housing and configured to create a flow of air, and a chamber coupled to the housing. The chamber is configured to accept a scent-releasing substance, such as a scent cartridge, and includes a plurality of openings. At least one of the openings is configured to input air into the chamber, while at least another one of the openings is configured to direct air from the chamber to the flow of air created by the air-moving device. The chamber further includes a user-controllable airflow adjustment device for controlling the size of at least one of the plurality of openings. In a preferred embodiment, the chamber includes a door that allows access to the chamber. The user-controllable airflow adjustment device can include, for example, a shutter or slidable panel to selectively adjust an effective surface area of one or more of the openings.

In a specific embodiment of the invention, the one or more openings configured to direct air from the chamber to the flow of air generated by the air-moving device is provided by one or more tubes with outlets disposed in the flow of air created by the air-moving device so as to draw air from the chamber using the Bernoulli effect.

In another aspect of the invention a method is provided for giving a scent-releasing capability to an appliance having an air-moving device disposed within a housing, in which the air-moving device is configured to create a flow of air. The method includes the step of coupling to the housing a chamber, in which the chamber is configured to accept a scent-releasing substance and has a plurality of openings. At least one of the openings is configured to input air into the chamber and at least another one of the openings is configured to direct air from the chamber to the flow of air created by the air-moving device. The chamber also includes a user-controllable airflow adjustment device for controlling the size of at least one of the plurality of openings. In a specific embodiment of the invention the method further includes causing the at least one of the openings to be disposed within the flow of air created by the air-moving device so as to draw air from the chamber using the Bernoulli effect.

### BRIEF DESCRIPTION OF THE DRAWINGS

The above and still further objects, features and advantages of the present invention will become apparent upon consideration of the following detailed description of a specific embodiment thereof, especially when taken in conjunction with the accompanying drawings wherein like reference numerals in the various figures are utilized to designate like components, and wherein:

FIG. 1 is a schematic view of an air-moving device with a scent-releasing mechanism in accordance with a first embodiment of the invention;

FIG. 2A is a front, left and elevated perspective view of an air-moving device with a scent-releasing mechanism in accordance with a second embodiment of the invention;

FIG. 2B is a magnified view of a portion of FIG. 2A;

FIG. 3 is a front, left and elevated perspective view of an air-moving device with a scent-releasing mechanism in accordance with a third embodiment of the invention; and

FIG. 4 is a front, left and elevated perspective view of an air-moving device with a scent-releasing mechanism in accordance with a fourth embodiment of the invention.

### DETAILED DESCRIPTION

Embodiments of the present invention provide an improved scent-releasing mechanism that can be used with an air-moving device. The scent-releasing mechanism is placed on or within a housing of the air-moving device such that the scent-releasing mechanism is able to controllably release scent into a stream of air generated by the air-moving device, thus dispersing the scent within a space such as a room. The shape and configuration of the scent-releasing mechanism may be designed to match or to accommodate the housing of the air-moving device, such as by fitting within a mounting location substantially within the housing or by being attachable at a mounting location on an exterior of the housing. The scent-releasing mechanism is fitted, attached, mounted or the like such that a control mechanism is accessible from the exterior of the air-moving device, and such that the scent source can be replaced by a user.

The air-moving device typically will include a fan, but embodiments of the invention are usable with other kinds of air-moving devices such as bellows, an impeller, etc. The air-moving device is often incorporated within an environmental-control appliance such as a dehumidifier, humidifier, etc.

The scent is initially provided in the form of a scent cartridge, scent pad, or the like. For the purposes of this disclosure, the term “scent cartridge” is used to indicate any form of carrier, cartridge, pad or otherwise that is used to hold, encapsulate or otherwise retain a scent-releasing substance; the scent cartridge itself may be made or formed, for example, from a scent-releasing substance, or the scent-releasing substance may be coated onto or disposed in the scent cartridge. In preferred embodiments of the invention, the scent cartridge typically contains a scent in a volatile or evaporative form, such as a volatile solution or an evaporative solid or gel.

The scent cartridge is received in a chamber. The chamber has a door or cover that may be opened and closed by the user in order to insert or to remove the scent cartridge. The chamber also has two or more relatively smaller openings, at least one being an input port that allows air to enter the chamber, and at least another opening being an output port that allows scented air to exit the chamber. At least one of these smaller openings can be controllably opened or closed in order to control air flowing from the input port through the chamber to the output port. For purposes of the following, the chamber is also intended to include any tube or the like that directs input and output airflows.

The control of the input or output port is by way of a user-operated control mechanism (e.g., handle, knob, shutter, slidable panel or the like) that is used to adjust an intensity of scent delivery. The user-operated control mechanism actuates a valve, shutter, baffle, or other aperture having an adjustable opening size, to control the opening size and thereby control the volume of air passing through.

The air which passes through the adjustable opening is then introduced into a stream of moving air produced by the air-moving device. The stream of moving air draws air from the chamber out through the output port by the Bernoulli effect, which in turn draws air through the input port and into the chamber so that it may then be expelled via the output port as scented air. A tube or the like may be used to help introduce air from the output port into the stream of moving air, and to introduce the air in a direction that facilitates drawing air from the tube. Adjusting the air flow through the scent chamber may affect the rate of evaporation of the scent solution. Increasing the rate of air flow through the chamber may increase the amount of scent released from the scent cartridge into the air flow from the appliance.

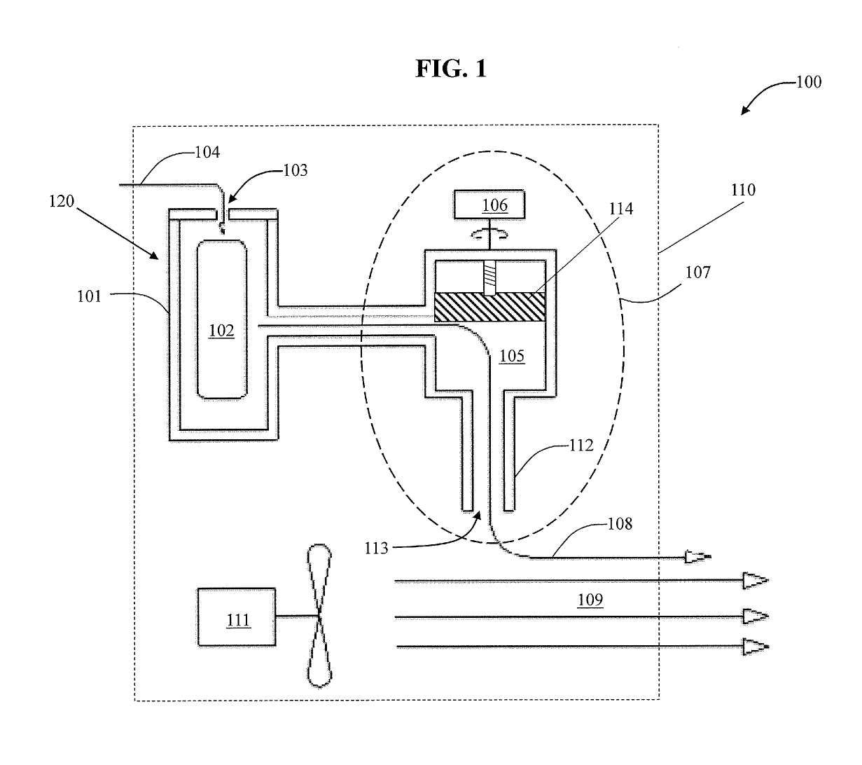

FIG. 1 illustrates a schematic view of an embodiment of the invention. An air-moving device 100 includes a scent-releasing mechanism 120 disposed within a housing 110 together with an air-moving device 111. Scent-releasing mechanism 120 includes a chamber 101 that is mechanically coupled to housing 110, either directly, indirectly through intermediate components, or formed as part of housing 110. Chamber 101 is configured to enclose a scent cartridge 102. Chamber 101 includes an input port 103, in the form of an aperture, which allows an input stream air 104 to enter chamber 101. Chamber 101 may include an adjustable output port 107, which includes an output aperture 113, and which is shown in FIG. 1 as further including a valve 105 that is controlled by a user-operated knob 106. The knob 106 is connected to a shutter or slidable panel 114 that increases or reduces the effective surface area of the output port 107 through which an output stream of air 108 can flow. In alternative embodiments (not illustrated in FIG. 1), input port 103 may include an adjustable aperture, or both input port 103 and output port 107 may include separately adjustable or co-adjustable apertures.

Referring again to FIG. 1, output port 107 may also include a tube 112 that is used to help introduce the output stream of air 108 into an air flow 109. Tube 112 makes it possible to dispose outlet aperture 113 within the air flow 109. The air flow 109 draws air from the chamber 101 out through outlet aperture 113 of output port 107 by the Bernoulli effect. The air flow 109 is produced by existing air-moving device 111 that is part of an appliance 100. Valve 105 together with knob 106 serves as an airflow adjustment device that controls the flux of air in output stream 108, and thus the intensity of scent release from the device 100.

Separately adjustable input ports 103 and output ports 107, each having a corresponding user-controllable airflow adjustment device, may be useful, for instance, if a first aperture is used to set the scent-releasing mechanism 100 to a predetermined intensity of scent release, and a second aperture is used to enable or disable scent release (e.g., by substantially fully opening or substantially fully closing the input port 103 or output port 107). Hence, one of the two user-controllable airflow adjustment devices may support a range of effective surface areas for its respective port 103, 107 that the user may select, from between fully open to fully closed; the other of the two user-controllable airflow adjustment devices may support only two settings for the effective surface area of its corresponding port 103, 107, being either fully open or fully closed.

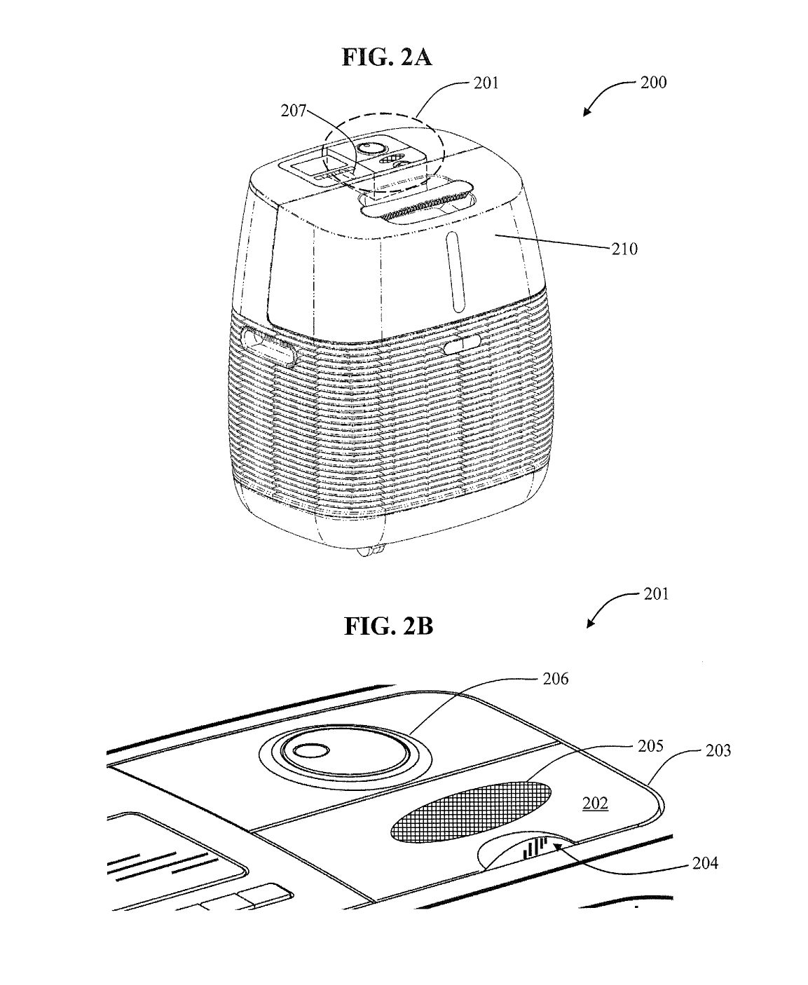

FIG. 2A illustrates a perspective view of an embodiment appliance 200. Portion 201 of appliance 200 is presented in a magnified view in FIG. 2B. An openable door 202 provides access to the chamber 203, and can be closed to substantially seal off chamber 203 but for the input and output ports. Door 202 can serve as part of the exterior of housing 210 of appliance 200. Door 202 is illustrated as including a user-operable portion 204 such as a handle, raised portion or the like in order to facilitate opening and closing of door 202. Other mechanisms can be used to facilitate opening and closing of door 202, such as a spring-loaded latch, magnets, etc. An input port 205 is formed in order to provide an opening into chamber 203. Chamber 203 is disposed within the exterior surface of housing 210 and may be formed, for example, contiguously as part of housing 210 or as a separate component coupled to housing 210. Input port 205 may be formed in door 202 as illustrated, or can be formed through a sidewall of chamber 203. Input port 205 may be shaped to provide a pleasing design. An output port 207 of chamber 203, disposed within housing 210 and thus normally not visible from the exterior of appliance 200, is shown in dotted lines for illustration purposes herein and leads to an air flow in the interior of appliance 200 that is produced by appliance 200. A knob 206, which is operable by a user, controls the opening size of the output port 207 from chamber 203, such as by a shutter, slidable panel or like, that selectively increases or decreases the effective surface area of output port 207; the effective surface area of output port 207 is the cross-sectional area of output port 207 that a flux of air can pass through as modified by the mechanism of knob 206. Typically the effective surface area of the output port 207 is determined by a subsection or region of the output port 207 that is directly affected by the mechanism controlled by knob 206 (i.e., a shutter, sliding panel or the like). Knob 206 and its related mechanism thus serve as an airflow adjustment device, controllable by a user, to control the intensity of scent released from device 200.

In operation of the embodiment of the invention 200, a user will open door 202 to insert a scent cartridge. When the user wants to introduce scent into a room containing appliance 200, the user will typically turn on appliance 200 (if not already on), thus producing an air stream from appliance 200. The user will then adjust knob 206 to control the effective surface area of the output port 207 until the desired amount or intensity of scent is released into an air stream produced by appliance 200. It will be appreciated that in other embodiments knob 206 could control the effective surface area of input port 205 to control the scent-releasing intensity of appliance 200.

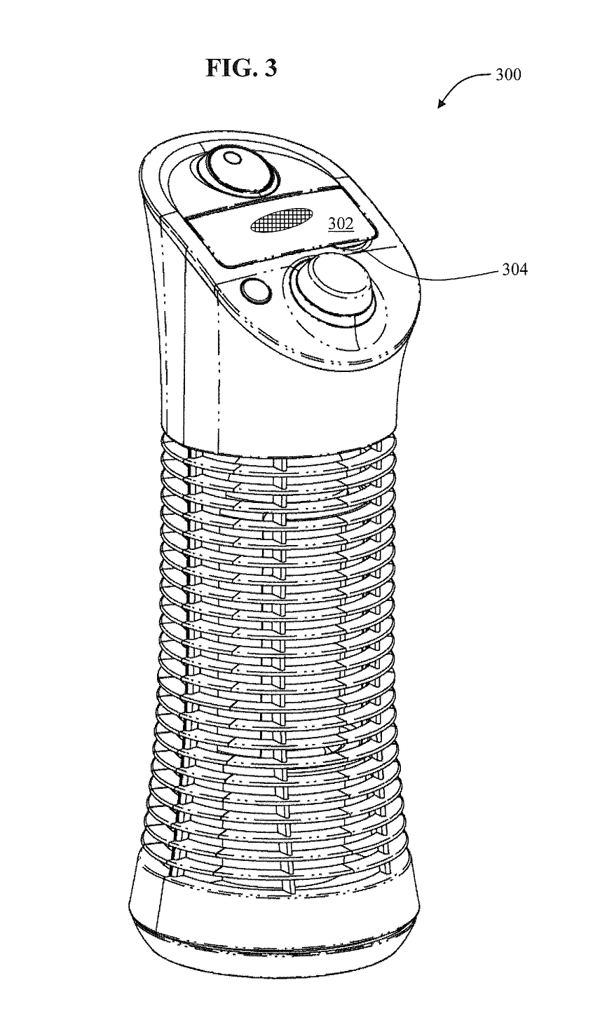

FIG. 3 illustrates a perspective view of an appliance 300 that incorporates an embodiment of the invention. In this embodiment 300, door 302 does not include a separate handle or the like. Rather, a portion 304 of the housing of appliance 300 is depressed, removed, or otherwise evacuated, thus exposing a side portion of door 302. The exposed side portion is a user-operable portion that can be used to open and close door 302.

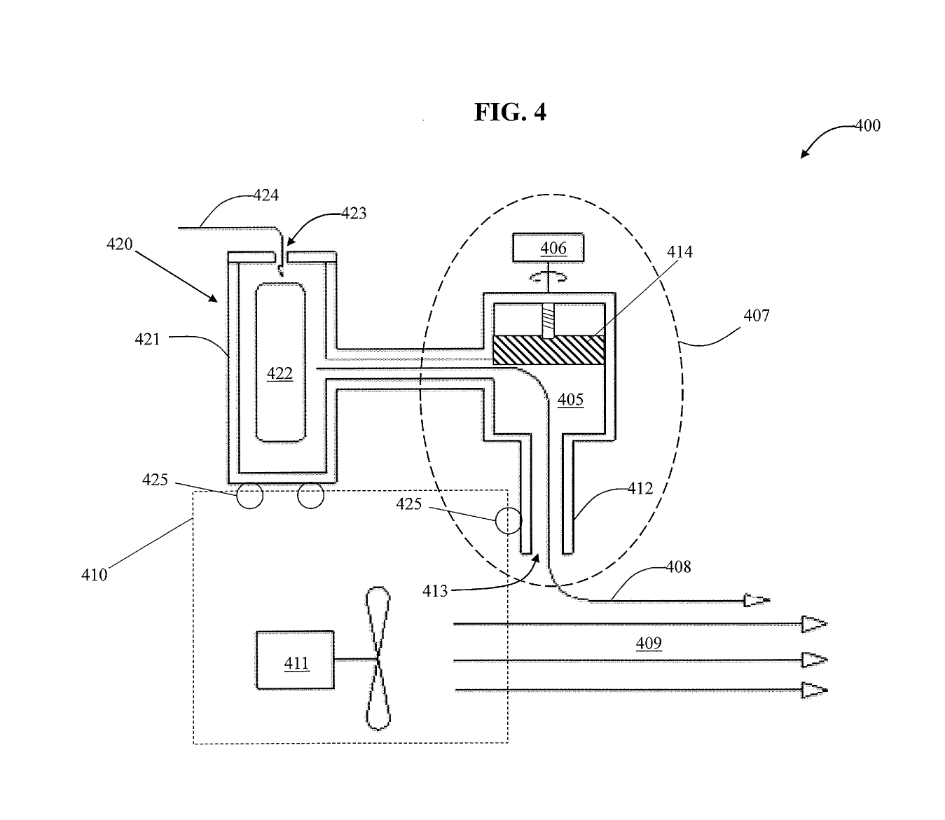

FIG. 4 illustrates a schematic view of a fourth embodiment of the invention 400. In this embodiment 400, a separate scent-releasing mechanism 420 is coupled to the housing 410 of the appliance 400 by way of one or more coupling mechanisms 425. Any suitable devices may be used for the coupling mechanisms 425, such as hooks, snaps, clamps, adhesives, welding points, etc. By way of example, the coupling mechanisms 425 may be configured to mechanically engage with suitable, corresponding features of the housing 410. An air-moving device 411 is disposed within the housing 410 and generates and output airflow 409 that exits from the housing 410. Scent-releasing mechanism 420 includes a chamber 421, which is externally coupled to housing 410 by way of the one or more coupling mechanism 425. Chamber 421 is configured to enclose a scent cartridge 422. Chamber 421 includes an input port 423, in the form of an aperture, which allows an input stream air 424 to enter chamber 421. Chamber 421 may include an adjustable output port 407, which includes an output aperture 413, and which is shown in FIG. 4 as further including a valve 405 that is controlled by a user-operated knob 406. The knob 406 is connected to a shutter or slidable panel 414 that increases or reduces the effective surface area of the output port 407 through which an output stream of air 408 can flow. Alternatively, valve 405 may be configured to regulate the airflow 424 into input port 423 by adjusting the affective surface area of input port 423. Or, both input port 423 and output port 407 may include separately adjustable or co-adjustable apertures.

Output port 407 is configured to be within output airflow 409 to take advantage of the Bernoulli effect provided by output airflow 409. For example, output port 407 may include a tube 412 that is used to help introduce the stream of air 408 exiting output aperture 413 into air flow 409 generated by air-moving device 411. Tube 412 may make it more convenient to dispose outlet aperture 413 within the air flow 409. Tube 412 may itself include a coupling mechanism 425 to couple to housing 410.

It will thus be seen that the objects set forth above, among those made apparent from the preceding description, are efficiently attained and, since certain changes may be made in the above construction(s) without departing from the spirit and scope of the invention, it is intended that all matter contained in the above description or shown in the accompanying drawing(s) shall be interpreted as illustrative and not in a limiting sense.

1-10. (canceled)

11. A scent-releasing device comprising:
a chamber configured to accept a scent-releasing substance, the chamber comprising:
an input aperture for accepting an input flow of air into the chamber;
an output aperture for outputting scented air from the chamber; and
a door for providing access to the chamber;
a first user-controllable airflow adjustment device for controlling an effective size of at least one of the input aperture and the output aperture; and
at least a connection device for coupling the chamber to an air-moving device.

12. The scent-releasing device of claim 11 further comprising an output port, the output port comprising a tube that provides the output aperture.

13. The scent-releasing device of claim 11 further comprising a second user-controllable airflow adjustment device for controlling a size of another of the at least one of the input aperture and the output aperture.

14. The scent-releasing device of claim 13 wherein the first user-controllable airflow adjustment device provides a user-selectable range of effective surface areas, and the second user-controllable airflow adjustment device provides only a substantially fully open position and a substantially fully closed position.

15. The scent-releasing device of claim 11 wherein the connection device is positioned with respect to the output aperture so that the output aperture is disposed in an airstream generated by the air-moving device.

16. A scent-releasing device comprising:
a casing configured to attach to an air-moving device and defining a chamber configured to accept a carrier comprising a scent-releasing substance, the chamber comprising:
an input aperture for accepting an input flow of air into the chamber;
an output aperture for outputting scented air from the chamber; and
a door for providing access to the chamber and sized to pass the carrier;
a first user-controllable airflow adjustment device for controlling an effective size of at least one of the input aperture and the output aperture; and
at least a connection device disposed on the casing for coupling the chamber to the air-moving device;
wherein the connection device is positioned with respect to the output aperture so that the output aperture is disposed in an airstream generated by the air-moving device.

17. The scent-releasing device of claim 16 further comprising an output port, the output port comprising a tube that provides the output aperture.

18. The scent-releasing device of claim 16 further comprising a second user-controllable airflow adjustment device for controlling an effective size of another of the at least one of the input aperture and the output aperture.

19. The scent-releasing device of claim 18 wherein the first user-controllable airflow adjustment device provides a user-selectable range of effective surface areas, and the second user-controllable airflow adjustment device provides only a substantially fully open position and a substantially fully closed position.

20. A method for providing an air-moving device with an adjustable scent-releasing capability, the method comprising:
obtaining the scent-releasing device of claim 16; and
utilizing the at least a connection device to connect the scent-releasing device to a housing of the air-moving device so that the output aperture is disposed in the airstream generated by the air-moving device.
